(19) United States
(12) Patent Application Publication
(10) Pub. No.: US 2008/0262499 A1
(43) Pub. Date: Oct. 23, 2008

(54) Self Retaining Acetabular Component Alignment Device for Total Hip Arthroplasty

(76) Inventors: Nicholas J. Giori, Stanford, CA (US); Eric Topp, Palo Alto, CA (US)

Correspondence Address: Nicholas John Giori, 1060 Vernier Place, Stanford, CA, 94305, US

(21) Appl. No.: 12/058,719

(22) Filed: Mar. 30, 2008

Related U.S. Application Data
(60) Provisional application No. 60/912,428, filed on Apr. 17, 2007.

(51) Int. Cl. A61F 5/00

(52) U.S. Cl. 606/87

(57) ABSTRACT

A spring-loaded, self-retaining, clamp like alignment device used to guide the placement of the acetabular component during total hip arthroplasty is disclosed. The device comprises two longitudinal elements: the first longitudinal element has a proximal end with a handle, a groove within and a spring loaded into the groove. The second longitudinal element has a distal end to which an acetabular component is attached and a mid-section that fits into the groove of the first longitudinal element and a proximal end with a knob. When the hook present at the distal end of the first longitudinal element is placed into the sciatic notch in the pelvic bone and the proximal end clipped to the mid-section of the second longitudinal element, the acetabular component at the distal end of the second longitudinal element is consistently positioned in the acetabulum at 45-degree abduction and 20-degree anteversion. While the surgeon impacts the acetabular component into place the spring located in the proximal end of the first longitudinal element maintains the proper alignment of the acetabular component and keeps the deep part of the clamp from injuring structures traversing the sciatic notch.

### CROSS REFERENCE TO RELATED APPLICATION

This application claims priority to U.S. provisional patent application No. 60/912,428 filed Apr. 17, 2007.

### FIELD OF THE INVENTION

This invention relates generally to alignment devices and particularly for those used to guide the acetabular component during total hip replacement surgery.

### DESCRIPTION OF THE RELATED ART

Total hip replacement surgery is one of the most common orthopedic procedures performed. It involves replacement of all of the diseased hip joint with an artificial device (prosthesis). Hip replacement surgery is performed about 200,000 times in the United States every year. This common procedure is performed to relieve hip pain associated with conditions such as osteoarthritis, inflammatory arthritis, and osteonecrosis.

The hip is a ball and socket joint composed of two parts. The socket (acetabulum) is part of the pelvis and the ball (femoral head) is the upper end of the thigh bone. In a total hip replacement procedure, a cup generally consisting of a metal shell and a bearing surface is inserted in the acetabulum. Also, the upper portion of the thigh bone is replaced with a metal stem with a ball at its upper end to fit into the acetabulum. The cup and stem attach to the pelvis and the thigh bone with fast-setting plastic cement or through bone in-growth, a process in which the bone grows into the replacement stem and cup.

One of the difficult parts of the total hip arthroplasty surgery is the consistent alignment of the acetabular component. When the acetabular cup is not correctly oriented, this malpositioning is correlated with increased risk and frequency of dislocation and impingement. Malpositioning of the acetabular component is also associated with late dislocation.

Positioning of the acetabular component using anatomical landmarks may reduce the incidence of dislocation from improper acetabular orientation. The pelvis provides bony landmarks (the anterior superior iliac spines and the pubic tubercles), which, when used to define the coronal plane of the pelvis, allows cup orientation in abduction and version.

Despite the anatomical landmarks that may serve as a guide during surgery, malposition of the acetabular component does occur. This is because these bony landmarks are never directly seen. Acetabular component orientation is thus difficult to assess accurately because soft tissues overlay these bony landmarks, and because patient positioning devices that are used to stabilize the pelvis during surgery further obscure these landmarks.

When the surgery is performed in the lateral decubitus position, which is by far the most common position utilized for total hip arthroplasty in the United States, it is extremely important to control pelvic position and ensure stability of the pelvis as the leg is manipulated during surgery. This is difficult to achieve and contributes to the variability seen in acetabular component positioning.

Although the position of the cup is important for the prognosis and function of the hip, most surgeons place the cup without any specific guidance devices that orient to the bony anatomy of the pelvis. In 99% of the instances, the surgeon visually estimates the position of the acetabular component based on its relationship to the trunk of the patient, which is obscured with surgical drapes, and decides if it meets the desired orientation before securing it. A very small percentage (about 1%) of the cases are performed using computer assisted positioning of the acetabular component, which is a technology that is, as yet, in its infancy.

In addition, most total hip arthroplasty procedures done in the United States are done by surgeons who do less than 10 total hip arthroplasties per year. This may further account for inconsistency in the accurate positioning of the acetabular component during surgery.

One of the routine methods to orient the cup is the free-hand positioning, with the patient in a standardized position and a cup positioner, with the patient and floor as reference. Specific mechanical alignment guides such as CT guided systems using computer assisted technology have been designed to add precision and accuracy.

Existing alignment jigs rely on reproducible patient positioning in the operating room—which is difficult to achieve. In certain situations X-rays can be used to align the implant. As can be understood, an alternate technique that does not rely on accurate patient positioning or on X-rays would be highly desirable, as each of these techniques have their own drawbacks.

There is therefore a need for an accurate positioning of the acetabular component at the time of surgery to prevent post-operative complication such as dislocation. Hence, a self retaining jig that self-aligns in a reliable and reproducible way using internal bony anatomy as a guide would be extremely useful to surgeons performing this procedure.

### SUMMARY OF THE INVENTION

The invention is an alignment device used to guide the placement of the acetabular component during total hip arthroplasty comprising of two parts. The first is a longitudinal element or an aligner with two ends, wherein the distal end is curved, and the proximal end has a means of coupling in a reproducible way to the insertion rod. The second is a longitudinal element or the insertion rod. The distal end of the insertion rod is designed to be connected to the acetabular component. The mid-section is designed to connect in a reproducible way to the other member, and the proximal end is designed for impaction.

In a preferred embodiment, the acetabular alignment device comprises a first and a second longitudinal element, where the distal end of the first longitudinal element or aligner is designed to hook around the sciatic notch and proximally attaches to the mid-section of the second longitudinal element or insertion rod. The insertion rod is an impaction device with the acetabular component attached to its distal end. Once the aligner and the insertion rod are assembled, the first longitudinal element aligns the acetabular component along a line defined by two points—the center of the inlet of the acetabulum and a point in the pelvis accessible through the sciatic notch that consistently orients the acetabular component in proper alignment of 45-degrees of abduction and 20-degrees of anteversion. After proper positioning, the acetabular component is impacted into place while the spring maintains the alignment and also keeps the deep part of the clamp from injuring structures traversing the sciatic notch.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 is a model of the pelvis, the alignment hook and insertion rod with the acetabular component at its distal end.

FIG. 2 shows the aligner with a spring loaded onto the groove

FIG. 3 shows the device when assembled—the alignment hook attached to the impaction device with the acetabular component at its distal end.

FIG. 4 shows the surgeon placing the hook into the sciatic notch during the surgery

FIG. 5 shows the two separate components, the hook and the acetabular component in their desired anatomical location before securing them together.

FIG. 6 is a picture of the components when assembled together, whereby the acetabular component is aligned to 45-degrees of abduction and 20 degrees of anteversion.

### DETAILED DESCRIPTION OF THE PREFERRED EMBODIMENTS

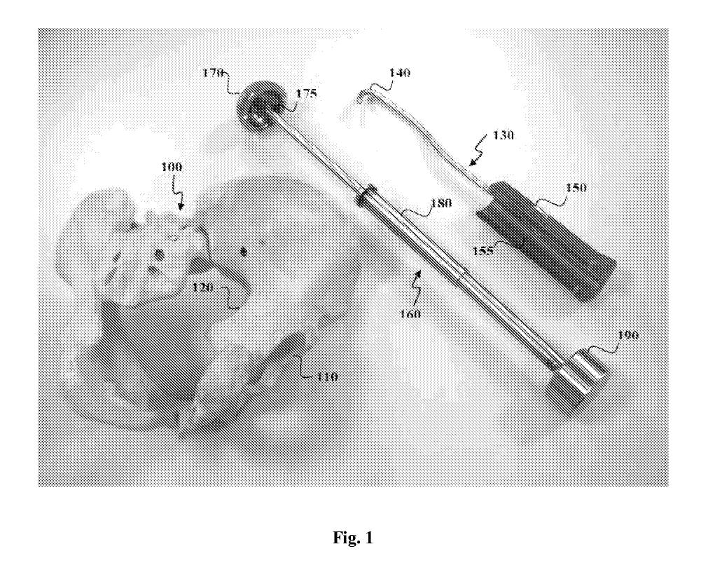

The invention is an alignment device used to guide the placement of the acetabular component during total hip arthroplasty. The device comprises a first longitudinal element (sometimes referred to as an alignment hook or aligner), and a second longitudinal element (sometimes referred to as the insertion rod or the impaction device) with an acetabular component at the distal end, as shown in FIG. 1. The pelvis 100 shows the relevant bony locations, acetabulum or acetabular inlet or the socket 110 and the sciatic notch 120. The aligner 130 has a curved portion or a hook 140 at its distal end and a handle 150 at its proximal end. The insertion rod 160 has the acetabular component 170 attached at its distal end, a thicker mid-section 180 and a flattened section 190 at its proximal end. The proximal end of the insertion rod is designed for impaction with a mallet and can take many shapes. The acetabular component 170 is coupled to the distal end 175 such that the acetabular component 170 can be easily detached from the insertion rod 160. The coupling mechanism could be any of those methods that are well known in the art for easy detachment of two coupled elements, such as a thread, clip or the like.

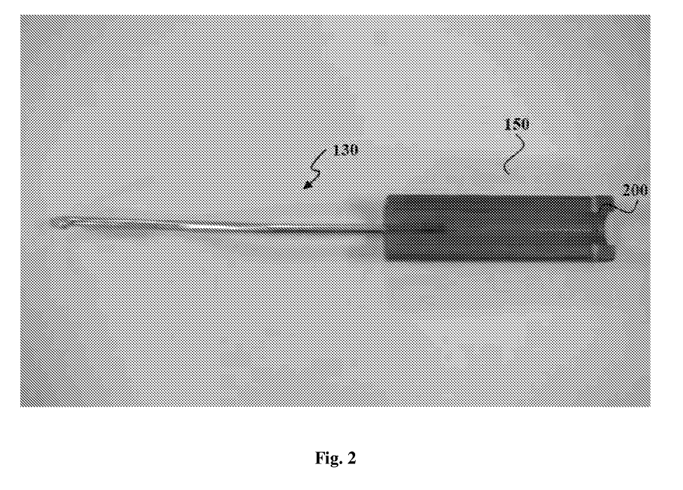

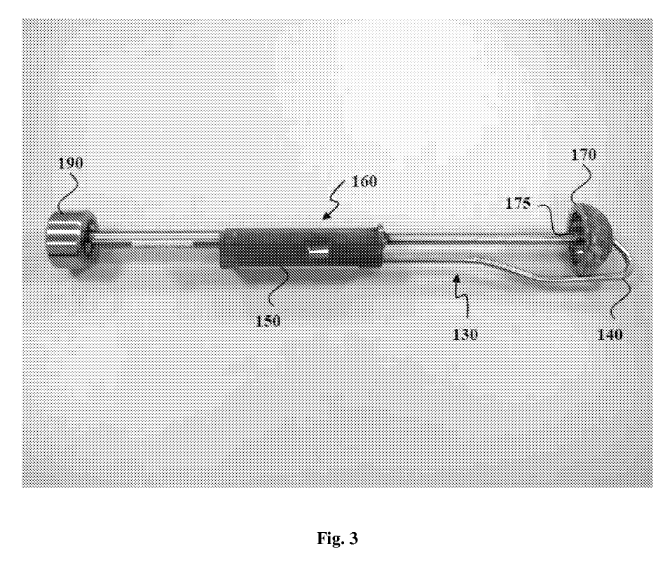

The proximal end of the first longitudinal element has a handle portion 150 with a groove 155 and a spring 200 loaded onto it (FIG. 2). The groove 155 accommodates the mid-section 180 of the second longitudinal element and secures the assembly. The spring 200 on the other hand maintains the alignment of the acetabular component and keeps the deep part of the hook from injuring structures traversing the sciatic notch 120. The grooved handle 150 of the first longitudinal element 130 when attached to the mid-section 180 of the second longitudinal element 160 aligns the acetabular component 170, as shown in FIG. 3.

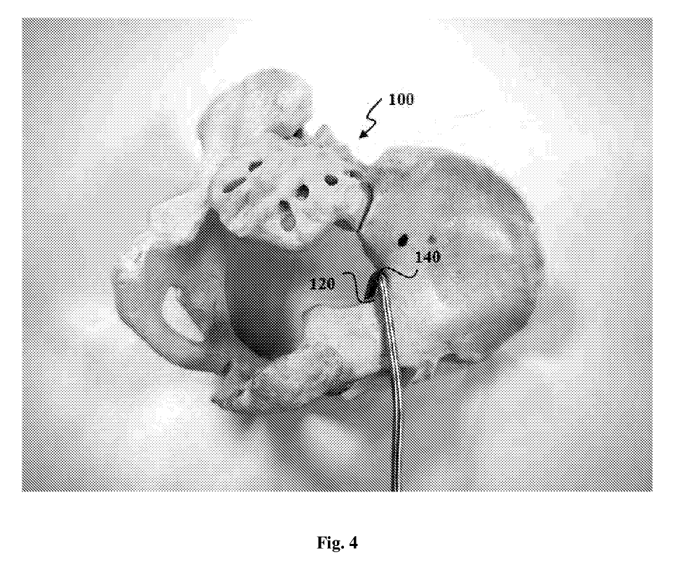

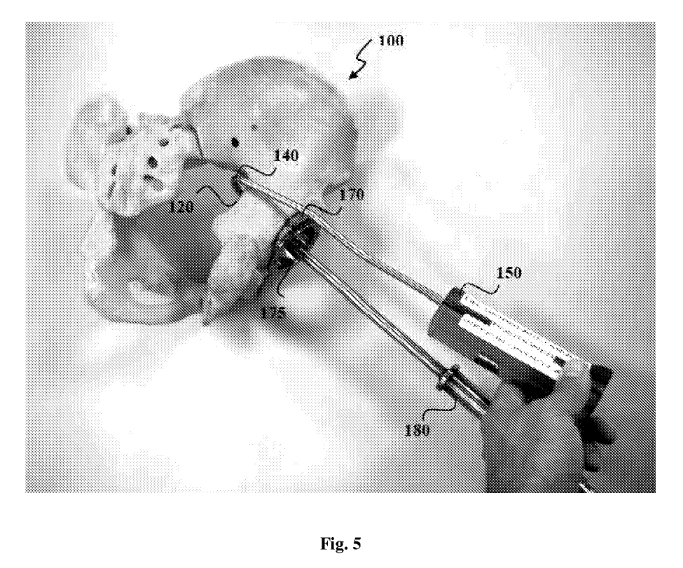

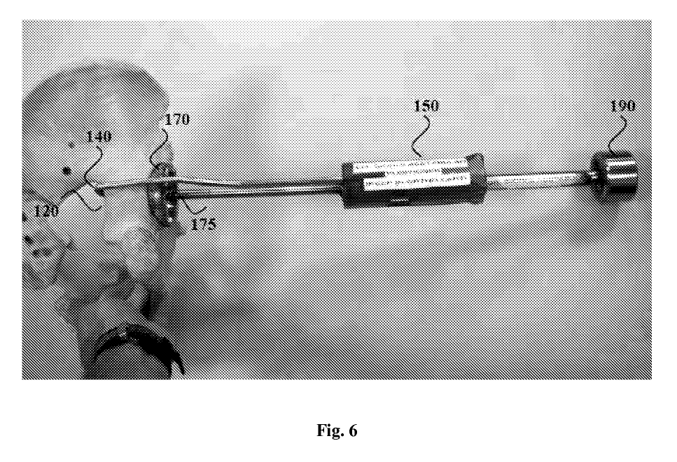

During surgery, the surgeon first exposes the acetabulum 110 and prepares the acetabular bed. The surgeon initially places the distal end of the first longitudinal element—the curved portion or hook 140—into the sciatic notch 120 of the pelvis (FIG. 4). The surgeon then places the acetabular component 170, which is at the distal end of the second longitudinal element, into the acetabulum 110. Then, the mid-section 180 of the second longitudinal element is attached along the groove 155 side of the handle 150 of the first longitudinal element, thus securing the assembly (FIG. 5). This arrangement further ensures that the first longitudinal element aligns the acetabular component along a line defined by two points—the center of the inlet of the acetabulum and a point in the pelvis accessible through the sciatic notch. This consistently orients the acetabular component in proper alignment of 45-degrees of abduction and 20-degrees of anteversion (FIG. 6). The surgeon then impacts the acetabular component 170 into the acetabulum 110 by hammering at the proximal end of the insertion rod 190. While doing so, the spring 200 loaded onto the groove 155 in the proximal end of the aligner maintains the alignment and also keeps the deep part of the hook from injuring structures traversing the sciatic notch.

While the invention has been disclosed with reference to certain embodiments, it will be understood by those skilled in the art that various changes may be made and equivalents may be substituted without departing from the scope of the invention. Particularly, in the area of computer-assisted surgery, a specially designed hooked probe can be used to identify the target point within the pelvis that the acetabular component can be aligned towards. This would obviate the need to register other points on the pelvis such as the anterior superior iliac spines and the pubic tubercles.

What is claimed is:

1. An alignment device for guiding the placement of the acetabular component during total hip arthroplasty comprising an aligner and an insertion rod, said aligner comprising a longitudinal member with two ends, wherein the distal end is adapted to engage with an anatomical landmark, and the proximal end is provided with means of coupling in a reproducible way to the insertion rod, said insertion rod comprising a longitudinal member with a distal end adapted to detachably hold the acetabular component, a midsection adapted to be releasably coupled to the proximal end of the aligner, and a proximal end adapted for impaction, whereby when the aligner is engaged with the anatomical landmark, the acetabular component is placed in the desired orientation.

2. The alignment device of claim 1, wherein the means of coupling the proximal end of the aligner to the insertion rod comprise a handle with a groove

3. The alignment device of claim 2, wherein a spring is loaded into the groove and becomes compressed when the aligner is coupled to the insertion rod, thus providing compression between the distal end of the aligner and the distal end of the insertion rod.

4. The alignment device of claim 3 wherein the midsection of the insertion rod has a thicker area adapted to accommodate the groove of the aligner

5. The alignment device of claim 1, wherein upon assembling the aligner and the insertion rod, pulling the aligner towards the user compresses the insertion rod.

6. The alignment device of claim 1, wherein the distal end of the aligner has a curved portion.

7. The alignment device of claim 6, wherein the curved portion is a hook.

8. The alignment device of claim 1, wherein the anatomical landmark is a sciatic notch.

9. A method of placing an acetabular component in desired orientation during hip replacement, comprising the steps of (a) attaching the acetabular component to a distal end of the insertion rod, (b) engaging a distal end of an aligner with an anatomical landmark, (c) inserting the acetabular component into the entrance of the acetabulum, (d) coupling a proximal end of the aligner with a mid-section of the insertion rod, thereby positioning the acetabular component in the desired orientation, (e) impacting the proximal end of the insertion rod thereby seating the acetabular component in place, and (f) detaching the acetabular component from the insertion rod.

10. The method of claim 9 wherein upon assembling the aligner with the insertion rod, pulling the aligner towards the user compresses the insertion rod.

11. The method of claim 9 wherein the aligner has a curved portion which engages with an anatomical landmark.

12. The method of claim 11 wherein the curved portion of the aligner is a hook.

13. The method of claim 9 wherein the anatomical landmark is a sciatic notch.

14. The method of claim 9, wherein when the aligner is engaged with the anatomical landmark, the acetabular component is aligned in the desired orientation.

15. The method of claim 9, wherein the acetabular component is detachably connected to the insertion rod.

16. The method of claim 9, wherein when the aligner and insertion rod are secured, the acetabular component is consistently aligned at 45 degrees of abduction and 20 degrees of anteversion.
